(19) United States
(12) Patent Application Publication
(10) Pub. No.: US 2025/0107884 A1
(43) Pub. Date: Apr. 3, 2025

(54) COMPOSITE TISSUE PRODUCT ANCHOR BOLSTER FOR THREE-DIMENSIONAL BIOLOGIC SCAFFOLDS AND RELATED METHODS

(71) Applicant: LifeCell Corporation, North Chicago, IL (US)

(72) Inventor: Dennis Y. Lee, Scotch Plains, NJ (US)

(21) Appl. No.: 18/792,639

(22) Filed: Aug. 2, 2024

Related U.S. Application Data
(63) Continuation of application No. 18/612,604, filed on Mar. 21, 2024.
Application No. 18/612,604 is a continuation of application No. 16/842,162, filed on Apr. 7, 2020, now abandoned.
(60) Provisional application No. 62/830,674, filed on Apr. 8, 2019.

(51) Int. Cl. A61F 2/12; A61L 27/36

(52) U.S. Cl. CPC A61F 2/12; A61L 27/362; A61L 27/3633; A61F 2220/0025; A61L 2430/04

(57) ABSTRACT

The present disclosure provides anchor bolsters for three-dimensional biologic scaffolds, including related methods of formation and use. The composite bolster-scaffold provides an improved device to securely anchor a three-dimensional tissue scaffold in position relative to surrounding anatomic structures, allowing time for colonization of native cells and subsequent incorporation of the three-dimensional tissue scaffold by the recipient tissue bed.

This application is a continuation of U.S. patent application Ser. No. 18/612,604, which was filed on Mar. 21, 2024, which is a continuation of U.S. patent application Ser. No. 16/842,162, which was filed on Apr. 7, 2020, which claims priority under 35 U.S.C. § 119 to U.S. Provisional Patent Application No. 62/830,674, filed Apr. 8, 2019. The contents of each of these applications are incorporated herein by reference in their entirety.

The present disclosure relates to tissue products, and, more particularly, to devices and methods for anchoring acellular tissue scaffolds to an anatomic structure of a recipient.

Biologically derived acellular tissue scaffolds are engineered to achieve many goals. The ability of tissue scaffolds to incorporate and promote tissue regeneration creates many clinical applications for these compositions. For example, acellular tissue scaffolds may be used to promote regeneration of tissue lost due to trauma, infection, ischemia, surgical resection of malignancy, and other causes. Acellular tissue scaffolds also have utility in aesthetic treatments and surgeries, including treatment of wrinkles, breast reconstruction or augmentation, and other tissue-augmentation procedures.

Tissue scaffolds can be used instead of or along with a synthetic implant. Like synthetic implants, acellular tissue scaffolds may be designed in virtually any shape, including two-dimensional flat sheets and three-dimensional forms. Tissue scaffolds having a three-dimensional component add volume and shape to the recipient's implantation site. Unlike synthetic implants, however, tissue scaffolds induce a minimal or absent host inflammatory response. Tissue scaffolds are less radiodense than synthetic implants, which can interfere with accurate interpretation of mammograms and other diagnostic radiological procedures performed post-implantation. Where synthetic implants are generally inert and do not promote regenerative or other favorable biologic activity in the recipient, tissue scaffolds mimic the extracellular matrix of the surrounding native tissue into which the scaffold is implanted. This property of extracellular tissue matrices may favorably induce cells at the implantation site, such as fibroblasts, adipocytes, myocytes, and other cell types, to transform the implanted tissue scaffold into a desired tissue type.

Consequently, three-dimensional tissue scaffolds have potential for use in many clinical applications. In one example, three-dimensional tissue scaffolds may be useful in post-mastectomy breast reconstruction or augmentation procedures. A tissue scaffold's decreased inflammatory response versus a conventional synthetic implant may mitigate long-term deformities arising from capsule formation and subsequent capsular contracture. This decreased inflammatory response is observed even in cases wherein a tissue scaffold is implanted with a synthetic implant.

Three-dimensional tissue scaffolds, however, may be improved by modifications to prevent undesired movements or provided additional structural support. Accordingly, composite tissue products with an improved anchoring bolster are desired.

Disclosed is a composite tissue product anchor bolster comprising a tissue scaffold and an anchor bolster securely coupled to the tissue scaffold. In some embodiments, the tissue scaffold is an acellular dermal matrix. In some embodiments, the tissue scaffold is an acellular adipose matrix. In some embodiments, the tissue scaffold is an acellular muscle matrix. In some embodiments, the tissue scaffold is a formed three-dimensional tissue scaffold.

The anchor bolster comprises an acellular dermal matrix, in some embodiments. In some embodiments, the anchor bolster comprises a synthetic product. The anchor bolster comprises an anchor fixation point, in some embodiments. The anchor bolster may be shaped as a tab, as a ribbon, or as a combination of tabs and ribbons.

Also disclosed is a tissue scaffold fixation system comprising a tissue scaffold, an anchor bolster coupled to the tissue scaffold, and an anchor, wherein the anchor couples the anchor bolster to an anatomic structure.

Disclosed is a method of using a composite tissue product anchor bolster comprising steps of selecting a tissue scaffold having a composite tissue product anchor bolster, positioning the tissue scaffold proximate to an anatomic structure, attaching a surgical anchor to the tissue product anchor bolster, and securing the anchor.

### BRIEF DESCRIPTION OF THE DRAWINGS

FIGS. 1A-B are stylized perspective views of example embodiments of a composite tissue product anchor bolster for a three-dimensional biologic scaffold;

FIGS. 2A-B are stylized perspective views of an alternative embodiment of a composite tissue product anchor bolster for a three-dimensional biologic scaffold;

FIG. 2C is a cross-sectional magnified view of an anchor bolster for a three-dimensional biologic scaffold;

FIG. 3 is a front perspective view of an additional embodiment of a composite tissue product anchor bolster for a three-dimensional biologic scaffold formed as a breast implant;

FIG. 4 is a cutaway view of a composite tissue product anchor bolster for a three-dimensional biologic scaffold formed as a breast implant; and

FIG. 5 is a schematic flow-chart diagram of a method of using a composite tissue product anchor bolster for a three-dimensional biologic scaffold.

### DETAILED DESCRIPTION OF EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this disclosure, the use of the singular includes the plural unless specifically stated otherwise. In this disclosure, the use of “or” means “and/or” unless stated otherwise. Furthermore, the use of the term “including,” as well as other forms, such as “includes” and “included,” is not limiting. Any range described herein will be understood to include the endpoints and all values between the endpoints.

The section headings used herein are for organizational purposes only and are not to be construed as limiting the subject matter described. All documents or portions of documents cited in this disclosure, including but not limited to patents, patent applications, articles, books, and treatises, are hereby expressly incorporated by reference in their entirety for any purpose.

Various human and animal tissues can be used to produce products for treating patients. For example, tissue products for regeneration, repair, augmentation, reinforcement, and/or treatment of human tissues that have been damaged or lost due to various diseases and/or structural damage (e.g., from trauma, surgery, atrophy, degeneration) have been produced. Such products can include, for example, acellular tissue matrices, tissue allografts or xenografts, and/or reconstituted tissues (i.e., at least partially decellularized tissues that have been seeded with cells to produce viable materials.)

A variety of tissue products have been produced for treating bone and soft tissues. For example, ALLODERM® and STRATTICE® (LIFECELL CORPORATION, Madison, NJ) are two dermal acellular tissue matrices made from human and porcine dermis, respectively. Although such materials are very useful for treating certain types of conditions, materials having different biological and/or mechanical properties may be desirable for certain applications. For example, ALLODERM® and STRATTICE® have been used in the surgical treatment of structural defects and to provide support to tissues for abdominal walls or in breast reconstruction. The mechanical and biological properties of tissue products make them well-suited for these and other uses.

The present disclosure provides disclosure of products, devices, and methods utilizing composite tissue product anchor bolsters coupled to a three-dimensional biologic scaffold. The bolster can allow anchoring of the three-dimensional biologic scaffold to an anatomic structure of a body. The products, devices, and methods may be arranged to provide improved methods of treatment when using composite tissue product anchor bolsters for three-dimensional biologic scaffolds. The bolster can be securely coupled to the tissue scaffold to allow secure fixation of the scaffold to anatomic structures.

For some indications, it is important to properly anchor an implant comprising a three-dimensional acellular tissue matrix to anatomic structures at the implantation site. Forces, including gravity, act on the implant and soft tissues proximate to the implant. If implant anchoring is inadequate, the implant may sag, rotate, or otherwise migrate from its intended position or orientation. Unintended movement can be problematic where the implant location is at an aesthetically significant anatomic region, such as the female breast. Migration of breast implants laterally, medially, superiorly, and inferiorly has been described. Additionally, when the implant is not symmetrical, such as a pear-shaped breast implant, rotation can also create a physical deformity. Some three-dimensional tissues, however, do not have adequate structural integrity to retain sutures, staples, or other surgical anchors, wherein the anchor pulls through the three-dimensional form. A three-dimensional soft tissue scaffold may not have sufficient density and tensile strength to hold sutures, surgical screws, staples, or other available surgical anchoring and fixation devices. A means for properly anchoring the tissue scaffold may, therefore, be desirable.

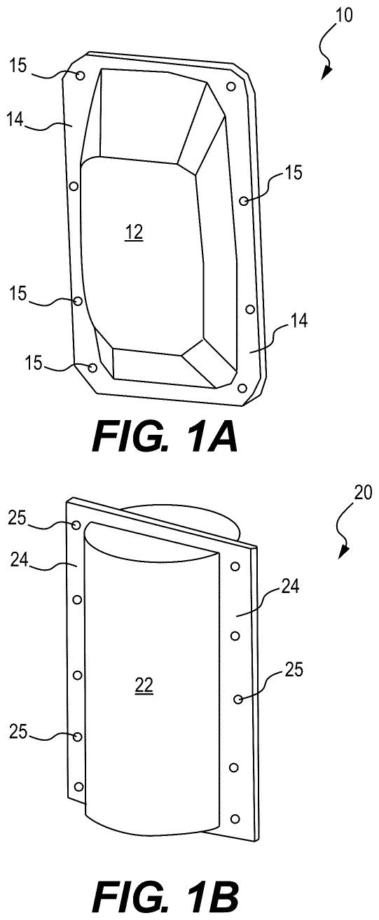

FIGS. 1A-B are stylized perspective views of example embodiments of a composite tissue product anchor bolster for a three-dimensional biologic scaffold. FIG. 1A shows a composite bolster-scaffold 10. Bolster-scaffold 10, in some embodiments, is a unitary body having a three-dimensional tissue scaffold 12 coupled to a surgical anchor bolster 14.

Three-dimensional tissue scaffold 12, in some embodiments, is a substantially acellular tissue matrix (“ATM.”) Types of ATM may include an ATM derived from a connective tissue, an adipose tissue, a muscle tissue, a cartilage tissue, or other soft tissues such as small intestine submucosal, bladder, stomach, or various layers of the GI tract. In some embodiments, tissue scaffold 12 is formed from fragments of an ATM mixed with a fluid, such as a slurry of fragments of an acellular adipose (or other tissue) tissue matrix fragments suspended in a slurry, wherein the slurry is placed into a mold and treated to retain a stable, three-dimensional shape having a porous, sponge-like structure. The tissue matrix sponge may resist deformation and loss of volume following implantation into a host. Tissue scaffold 12 may be used for regeneration, repair, replacement, or augmentation of a soft tissue, such as, for example, breast tissue.

In some embodiments, surgical anchor bolster 14 is formed from an intact acellular tissue matrix. As used herein, the term “intact acellular tissue matrix” refers to an extracellular tissue matrix having a shape and form substantially similar to the tissue from which the matrix is derived, although it will be understood that production of the acellular matrix (e.g., by removing cells) will produce a matrix that is modified from the original tissue matrix, by for example, changing the microstructure of the matrix. For example, an “intact acellular tissue matrix” produced from elongated, sheet-like tissue such as dermis, bladder, intestinal layer(s), stomach layer(s), dura, pericardium, or fascia may be in the form of a sheet. Such “intact acellular tissue matrices,” however, can include openings, meshes, or holes, as discussed further below, and may be modified, e.g., by cross-linking, enzymatic treatment, or chemical modification. “Intact acellular tissue matrices” would not include tissues that have been ground, cut, homogenized, or otherwise processed to form small tissue fragments or particles, even if such fragments or particles are resuspended or otherwise processed to produce a sheet or other form, formed from a connective tissue, in some embodiments.

Connective tissue and structures largely comprised of connective tissue, for the purposes of this disclosure, include skin, parts of skin (e.g., dermis), fascia, muscle (striated, smooth, or cardiac), pericardial tissue, dura, umbilical cord tissue, placental tissue, cardiac valve tissue, ligament tissue, tendon tissue, blood vessel tissue such as arterial and venous tissue, cartilage, bone, neural connective tissue, urinary bladder tissue, ureter tissue, and intestinal tissue. For example, a number of biological scaffold materials that may be used for the surgical anchor bolster 14 are described by Badylak et al., Badylak et al., “Extracellular Matrix as a Biological Scaffold Material: Structure and Function,” Acta Biomaterialia (2008), doi: 10.1016/j.actbio.2008.09.013. Suitable human and porcine dermal materials include, for example, ALLODERM® and STRATTICE®, respectively. In some embodiments, the ATM forming anchor bolster 14 is derived from porcine connective tissue. In some embodiments, the ATM forming anchor bolster 14 is derived from human connective tissue.

In some embodiments, tissue scaffold 12 and surgical anchor bolster 14 are formed as a composite unitary body. Methods of forming the composite unitary body are described in U.S. patent publication no. 2018-0008745, the disclosure of which is incorporated entirely herein by reference. Anchor bolster 14 may be coupled between layers of tissue scaffold 12, in some embodiments. In some embodiments, scaffold 12 and anchor bolster 14 are coupled together after each is formed separately. Methods for coupling scaffold 12 and anchor bolster 14 include creating a first surface feature on a surface of scaffold 12 surface that engages with a complementary second surface feature on a surface of anchor bolster 14. In some embodiments, the first surface feature is comprised by an exterior surface of scaffold 12. In some embodiments, the first surface feature is an internal surface, wherein anchor bolster 14 is embedded in the substance of scaffold 12 and wherein the interface between the second surface feature of anchor bolster 14 with the substance of scaffold 12 defines the first surface feature of scaffold 12. In some embodiments, other coupling means may be used, either in place or in addition to complimentary surface features, secondary surface features, and the like. Some examples of other coupling means include the use of biologic adhesives (e.g., enzymes, fibrin glue), non-biologic biocompatible adhesives (e.g., methyl methacrylate and other methacrylate and poly-methacrylate adhesives), biocompatible mechanical coupling means (e.g., sutures, screws, pins) and the like.

Some non-limiting examples of complimentary engaging surface features, designated by “first surface feature-second surface feature” include ridges-grooves, protrusion-dimple, peg-hole, and the like. In some embodiments, the first surface feature, the second surface feature, or both may comprise smaller, secondary surface features, such as textures or surface irregularities that increase friction between first surface feature engaged with second surface feature, wherein disengagement of the engaged first and second surface features is resisted by friction between the secondary surface feature(s).

In the example embodiments shown in FIG. 1A, anchor bolster 14 is coupled to an external surface of tissue matrix 12. The external surface is hidden beneath anchor bolster 14 and not shown in FIG. 1A.

In some alternative embodiments, an anchor bolster 24 is formed as a generally planar body passing through the substance of a tissue scaffold 22, such as the example embodiments shown in FIG. 1B, and in some other embodiments. In this and some related embodiments, anchor bolster 14 or anchor bolster 24 is formed as a “ribbon” around a perimeter of tissue scaffold 12 or tissue scaffold 22, according to the particular embodiment. The stylized embodiment shown in FIG. 1B, and related embodiments, can be used for applications wherein three-dimensional tissue scaffold 22 is formed to extend both deep and superficial to an anatomic structure to which product 22 is anchored. Examples of such applications include the axilla, the femoral triangle, and the neck. Wherein anchor bolster 24 is coupled to a fascial layer in these anatomic regions, tissue scaffold 22 may both fill a space or defect deep to the fascia to protect neurovascular structures and also re-establish normal soft tissue contours superficial to the fascia after disfiguring soft tissue loss from excision, infection, or other causes.

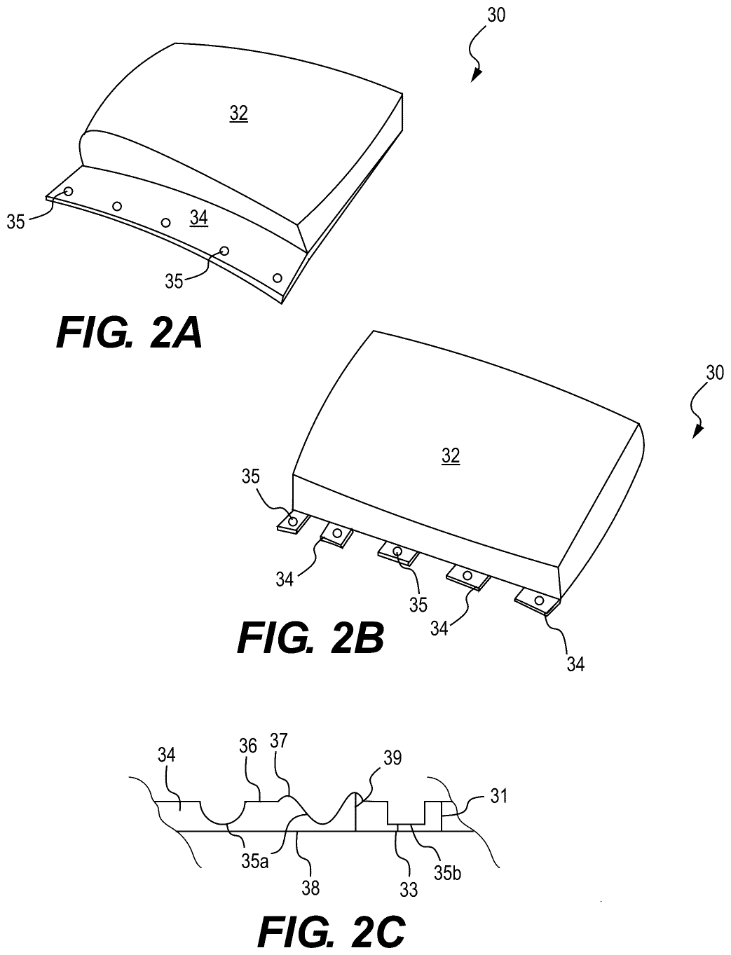

Tissue product anchor bolster 14, bolster 24, bolster 34, or bolster 44 can comprise an acellular matrix (e.g., dermal matrix) formed as a composite acellular tissue matrix with a three-dimensional tissue scaffold 12, scaffold 22, scaffold 32, and scaffold 42. In the example embodiments shown by FIG. 1A, anchor bolster 14 is configured in a form of a ribbon around the perimeter of a surface of scaffold 12. This is not, however, meant to be limiting. In some embodiments, anchor bolster 14 includes more than one anchor bolsters 14 disposed at intervals along the perimeter of scaffold 12. Surgical anchor bolster 14 is formed into any number of shapes, according the anticipated clinical use of scaffold 12. For example, anchor bolster 14 may be formed as a “tab” disposed at intervals along the perimeter, as shown by FIG. 2B, discussed herein below. In some embodiments, anchor bolster 14 is formed to anchor scaffold 12 in a subcutaneous position, such as to allow attachment of the clavipectoral fascia overlying the pectoralis major muscle and chest wall of a patient. In other embodiments, bolster 14 is formed to anchor scaffold 12 in a sub-pectoral location, such as to allow attachment of the chest wall at the inframammary fold and the chest wall lateral to the pectoralis major muscle. Attaching bolster 14 to the chest wall may prevent migration of scaffold 12 inferiorly on the chest wall, lowering the inframammary fold. Attaching bolster 14 may also prevent migration of scaffold 12 laterally into the axilla. Attaching bolster 14 to the chest wall, or other suitable anatomic structure, is performed to stabilize scaffold 12, thus allowing time for permanent fixation of scaffold 12 in position by incorporation into the tissue of the recipient. Other configurations of tabs, ribbons, and related forms of anchor bolster 14 are coupled to tissue scaffold 12 for reconstruction of the female breast or treatment of a bone or soft tissue defect at a specific anatomic site.

In some embodiments, surgical anchor bolster 14 is formed from a biocompatible synthetic material, such as polytetrafluoroethylene, polyethylene, polypropylene, polyester, silicone, and other biocompatible synthetic materials.

FIGS. 2A-B are stylized perspective views of additional embodiments of a composite bolster-scaffold for a three-dimensional biologic scaffold. As shown by FIGS. 2A-B, a composite bolster-scaffold 30 comprises a unitary body having a three-dimensional tissue scaffold 32 coupled to an anchor bolster 34. Anchor bolster 34 is shown with a plurality of anchor fixation points 35, wherein each anchor fixation point 35 is a generally circular hole. As mentioned herein, the shape of anchor fixation point 35 is not meant to be limiting. Anchor fixation points 35 may be located along one or more edges of the bolster-scaffold 30.

Additionally, in some embodiments, anchor bolster 34 does not comprise anchor fixation point 35. Generally an acellular dermal matrix formed from a porcine tissue is thicker and more resistant to penetration with a suture needle or other surgical anchor fixation device than an acellular dermal matrix formed from a human tissue. A human-derived acellular dermal matrix, much like human skin, holds suture well, is pliant, and minimally resists passage of a suture needle or suture through the tissue matrix material. Porcine-derived acellular dermal matrix, however, is dense, less pliant, and may present considerable resistance to perforation with a suture needle and suture. Accordingly, anchor bolster 34 formed from a porcine-derived dermal matrix may preferentially comprise a plurality of completely penetrating anchor fixation points 35, versus an anchor bolster 34 formed from a human acellular dermal matrix which may comprise a plurality of non-perforated anchor fixation points 35, or have no anchor fixation point 35. In some embodiments, composite tissue product anchor bolster 30 comprises both an anchor fixation point 35 fully penetrating anchor bolster 34 and an anchor fixation point 35 not fully penetrating anchor bolster 34, such as a dimple or a countersink, for example.

As shown by FIG. 2B, bolster-scaffold 30 comprises a discontinuous plurality of anchor bolsters 34, in some embodiments, anchor bolsters 34 may have a generally rectangular tab-shape, as shown in FIG. 2B. In some embodiments, anchor bolsters 35 may have a curvilinear shape. In some embodiments, the anchor bolsters 34 may include at least one having a tab-shape and at least one having a curvilinear shape. In some embodiments, anchor bolster 34 comprises one anchor fixation point 35. In some embodiments, anchor bolster 34 comprises a plurality of anchor bolster fixation points 35.

A porcine-derived acellular dermal matrix may be dense and somewhat more difficult to penetrate with a suture needle, compared to a human-derived acellular dermal matrix which is subjectively less dense, easier, and faster to pierce with a suture needle. Accordingly, in some embodiments, including the embodiment shown in the drawing figures discussed herein, surgical anchor bolster 34 has a pre-formed fixation point 35 configured such that a surgeon may more easily and expediently pass a suture needle, a tissue staple, a screw, or other surgical anchor through anchor bolster 34. In some embodiments, fixation point 35 is an opening between a first surface of the anchor bolster and a second surface of the anchor bolster and is formed as a hole, a slit, a fenestration, or similar opening, for example. In some alternative embodiments, fixation point 35 is not an opening between the first surface and the second surface of the anchor bolster, but a thinned area between the first surface and the second surface. For example, in some embodiments, fixation point 35 is a dimple, a depression, an indentation, a divot, a concavity, a dent, a pit, a countersink, or the like.

A three-dimensional tissue scaffold typically has limited tensile strength. Sutures or other surgical anchors can “pull through” the tissue scaffold, thereby failing to hold the tissue scaffold in place until such time as it becomes fully incorporated into and assimilated with the recipient's surrounding tissue. A substantially flat anchor bolster 34 formed from an acellular dermal matrix, or other connective tissue matrix instead of an adipose matrix or a muscle matrix, is denser and has a higher tensile strength than a three-dimensional tissue scaffold derived from non-dermal tissue. In some embodiments wherein additional strength is desired, fixation point 35 comprises a rim of additional acellular dermal matrix wherein the ribbon surrounding fixation point 35 is thicker than the surrounding anchor bolster 34. A thickened rim adds additional tensile strength to fixation point 35 at points whereupon the suture or other surgical anchor contacts fixation point 35.

FIG. 2C is a cross-sectional magnified view of an anchor bolster for a three-dimensional biologic scaffold. FIG. 2C shows different forms of an fixation point 35, including a fixation point 35a and a fixation point 35b. A porcine-derived acellular dermal matrix may be dense and difficult to penetrate with a suture needle, compared to a human-derived acellular dermal matrix which is subjectively less dense, easier, and faster to pierce with a suture needle. Accordingly, in some embodiments, including the embodiment shown in the drawing figures discussed herein, surgical anchor bolster 34 has a pre-formed fixation point 35 configured such that a surgeon may more easily and expediently pass a suture needle, a tissue staple, a screw, or other surgical anchor through anchor bolster 34. In some embodiments, fixation point 35 is an opening extending through a first surface 36 of anchor bolster 34 and a second surface 38 of anchor bolster 34. In these and some related embodiments, fixation point 35 is formed as a hole, a slit, a fenestration, or similar opening, for example. In some alternative embodiments, including the embodiment shown in FIG. 2C, fixation point 35 is not an opening extending through first surface 36 and second surface 38 of anchor bolster 34, but a thinned area of bolster 34 between first surface 36 and second surface 38. For example, in some embodiments, anchor bolster 34 comprises an fixation point 35a that is a depression with sloping sides, such as a dimple, depression, concavity, or dent. In some embodiments, anchor bolster 34 comprises an fixation point 35b that is a depression with straight sides, such as a divot, a pit, or a countersink. Other shapes and cross-sectional configurations are possible for fixation point 35, according to embodiments of the invention.

FIG. 2C shows anchor bolster 34 having a first thickness 31. In embodiments wherein fixation point 35 does not comprise an opening across two surfaces of anchor bolster 34, anchor bolster 34 has a second thickness 33 that is a thickness less than first thickness 31.

Also shown in FIG. 2C is a rim 37. Rim 37 is present in some embodiments wherein anchor bolster 34 has an fixation point 35 and may be present in such embodiments wherein fixation point 35 is a hole, a perforation, a slit, a penetration, a dimple, a pit, a countersink, or the like, without limitation. Rim 37 is positioned proximate to fixation point 35 and comprises a third thickness 39, as shown in FIG. 2C. Rim 37 provides additional mechanical strength around a perimeter (not shown) of fixation point 35, such that rim 37 resists “pull-through” of a suture, or other surgical anchor. Rim 37 is raised above first surface 36 at a location of anchor bolster 34 proximate to fixation point 35 having a third thickness 39, as shown by FIG. 2C.

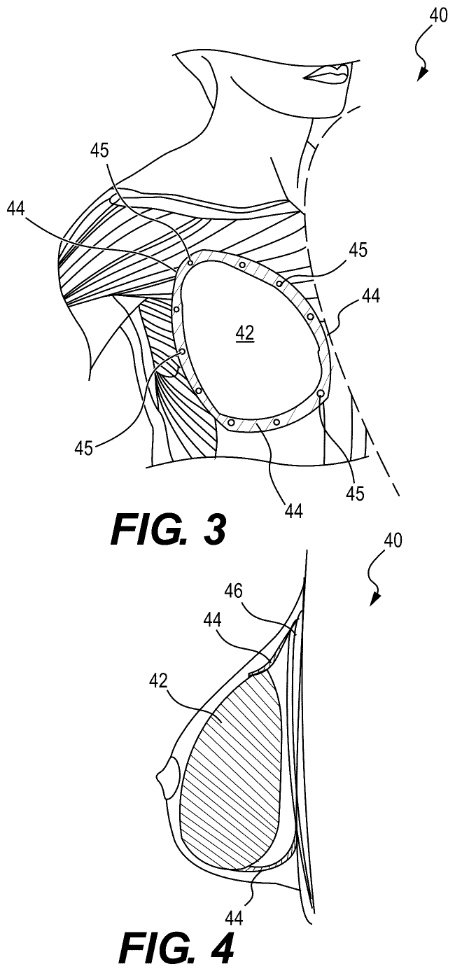

FIG. 3 is a front-perspective view of an additional embodiment of a composite tissue product anchor bolster for a three-dimensional biologic tissue scaffold. FIG. 3 shows a composite bolster-scaffold 40 comprising a tissue scaffold 42 in the shape of a female breast.

This form of composite tissue product anchor bolster is useful, for example, in breast reconstruction and augmentation procedures. This example, however, is not meant to be limiting. Tissue scaffold 42 may be pre-formed or cut to shape and size during surgery. Tissue scaffold 42 is formed into a variety of generally solid, three-dimensional shapes conforming to a shape or size according the intended implantation site of composite bolster-scaffold 40 on a particular patient. Some non-limiting examples of such applications include soft tissue and mixed-tissue reconstruction of tissue defects resulting from trauma, severe soft-tissue infection, tissue necrosis arising from correctable cardiovascular or vascular insufficiency, exposure to external beam radiation, and the like. For example, tissue scaffold 42 may be formed in the shape of an irregular pyramid to cover and protect the vascular structures of the femoral triangle following trauma, surgical debridement of infection, or resection in conjunction with a radical inguinal lymph node dissection. Many other shapes are possible, according to the anatomic location of the tissue defect. Tissue scaffold 42 is formed to fill a specific soft tissue defect, or to augment soft tissue in a particular anatomic area in an individual recipient prior to implantation, in some embodiments. The female breast-shape as shown by FIG. 3, therefore, is by way of example only.

Tissue scaffold 42 is coupled to an anchor bolster 44, as shown in FIG. 3. Similar to the example embodiment of anchor bolster 14 shown in FIGS. 1A-B, anchor bolster 44 is formed as a ribbon encircling a perimeter of three-dimensional tissue scaffold 42. In some alternative embodiments, anchor bolster 44 comprises a plurality of tabs positioned at intervals along a perimeter or tissue scaffold 42, similar to anchor bolster 34 of FIG. 2B. In some embodiments, including the embodiment shown in FIG. 3, anchor bolster 44 is shaped as a single, generally elongated ribbon coupled to a section of the perimeter of tissue scaffold 42 proximate to the inferior and lateral aspects of a breast reconstruction or augmentation site on a pectoralis muscle complex (chest wall) 46, as shown. In this configuration, and in some other embodiments, anchor bolster 44 acts both to fix tissue scaffold 42 in position and to define the inferior border (inframammary fold) and lateral border of the reconstructed or augmented breast. Wherein composite bolster-scaffold 40 is placed in a subcutaneous position, as in the example shown by FIG. 3, anchor fixation points 45 are attached to the clavipectoral fascia covering the pectoralis major muscle anteriorly, the serratus anterior muscle laterally, and the latissimus muscle posteriorly (anatomic structures illustrated but not labeled). Anchor bolster 44 is configured to provide necessary structural support, according to the size, shape, and anatomic site of implantation of the three-dimensional tissue scaffold.

In some embodiments, anchor bolster 44 comprises an anchor fixation point 45. Similar to anchor fixation point 15, discussed herein above, anchor fixation point 45 may be an opening between two surfaces of anchor bolster 44. In some other embodiments, anchor fixation point 45 is a thinning of anchor bolster 44 between the two surfaces, without forming a complete opening through the two surfaces of anchor bolster 44.

FIG. 4 is a cutaway view of a composite tissue product anchor bolster for a three-dimensional biologic scaffold formed as a breast implant. FIG. 4 shows composite bolster-scaffold 40 having tissue scaffold 42 coupled to anchor bolster 44. Tissue scaffold 42 is shown in cross-section in situ, following implantation into the subcutaneous space of the anterior chest wall of a recipient. Two sections of anchor bolster 44 are seen, a first section superiorly and a second section inferiorly. The first section of bolster 44 is anchored to the fascia overlying a pectoralis muscle complex 46 (anchors not shown), suspending a superior portion of tissue scaffold 42. Similarly, the second section of bolster 44 is shown supporting an inferior portion of tissue scaffold 42, anchoring tissue scaffold 42 to the inferior-anterior chest wall. Anchor bolster 44 is formed in accordance with the intended use of composite bolster-scaffold 40, whether to reconstruct or augment the female breast, or for other soft tissue reconstruction or augmentation applications in other areas of the body. In some embodiments, anchor bolster 44 comprises a ribbon-like configuration, similar to that shown in FIG. 3, wherein the ribbon is incomplete and only coupled to a portion of a perimeter of tissue scaffold 42, combined with one or more additional tab-shaped anchor bolsters 44.

There are many forms of surgical anchors that are suitable for use with composite bolster-scaffolds 10, 20, 30, and 40. Some non-limiting examples include sutures, including permanent non-absorbable and absorbable sutures. Surgical staples of various sizes and shapes may be used in conjunction with a composite bolster-scaffold. Screws, clips, bone-suture anchors, and the like may be used, according to the site of implantation of the composite bolster-scaffold.

Absorbable sutures, or other absorbable surgical anchors, are used in some embodiments, wherein ingrowth of and eventual replacement by a recipient's native connective tissue obviates the need to permanently anchor the tissue scaffold to surrounding soft tissue. Three-dimensional acellular tissue scaffolds are designed as a “scaffold” for a recipient's tissue to regenerated lost bone or soft tissue. Ideally, over time the tissue scaffold assimilates fully with the host's tissue. Following full incorporation, assimilation, remodeling, and replacement, there may be no further need to anchor a previously implanted tissue scaffold in position.

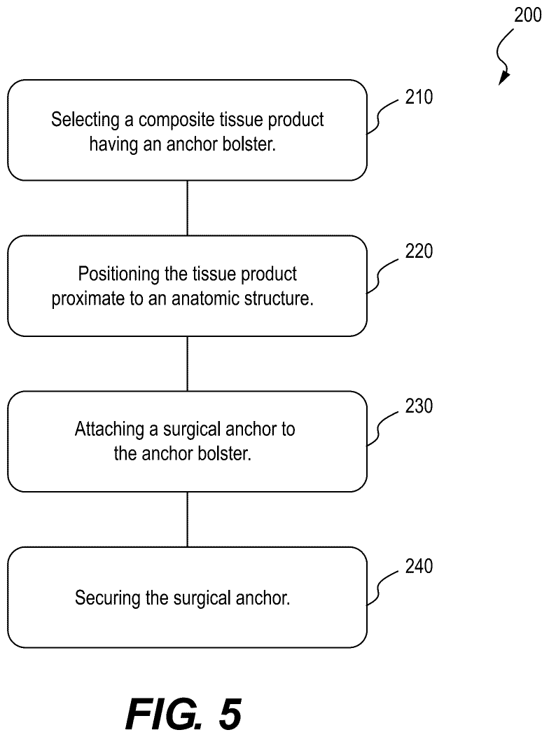

FIG. 5 is a schematic flow-chart diagram of a method of using a composite bolster-scaffold. FIG. 5 shows a method 200 of using a composite bolster-scaffold comprising a selecting step 210, a positioning step 220, an attaching step 230, and a securing step 240.

Selecting step 210, in some embodiments, comprises selecting a composite tissue scaffold having an anchor bolster. The three-dimensional tissue scaffold is chosen according to the intended surgical application. In some embodiments, the tissue scaffold is formed into a stable, three-dimensional shape prior to the implantation procedure. In some embodiments, the tissue scaffold is cut, compressed, or otherwise sized and shaped by trimming at, or proximate to, the time of implantation. In some embodiments, a standard-sized tissue scaffold is used. In some embodiments, the three-dimensional tissue scaffold is formed to generally conform to the dimensions of a soft tissue defect in an individual patient. In some embodiments, the size and shape of the soft tissue defect is determined by noninvasive imaging and computer-assisted modeling techniques to form a custom-size and shaped implant for a specific soft tissue defect in an individual patient.

Positioning step 220, in some embodiments, comprises positioning the tissue scaffold proximate to an anatomic structure. Positioning step 220 includes adjusting the three-dimensional tissue scaffold to an orientation of the soft tissue defect such that the tissue scaffold substantially fills the tissue defect without leaving empty space and without compressing the tissue scaffold or deforming the surrounding soft tissue. Positioning step 220 also comprises positioning an anchor bolster in contact with an anatomic structure to which the bolster is to be anchored. The anatomic structure is a fascia, a muscle, a bone, a connective tissue, or the like, in some embodiments.

Attaching step 230, in some embodiments, comprises attaching a surgical anchor to the anchor bolster. The surgical anchor is a suture, a screw, a stable, a clip, a bone suture anchor, or the like. In some embodiments, the surgical anchor may be an adhesive, such as a synthetic adhesive or a fibrin glue. The synthetic adhesive may be a polymethylmethacrylate glue or related, inert adhesive commonly used in joint replacement surgery and related procedures. In some embodiments, surgical anchors are attached via anchor fixation points located on the tissue scaffold or anchor bolster.

Securing step 240, in some embodiments, comprises securing the surgical anchor to the anchor bolster and to the anatomic structure. It may be useful, in some embodiments, to attach all surgical anchors to all anchor bolsters, or all anchor fixation points prior to securing the anchors. This technique allows for the anchors to be secured and tightened with the proper amount of tension to secure the device to the surrounding soft tissue without mal-positioning the tissue scaffold or causing deformation of the surrounding soft tissue.

The above description and embodiments are exemplary only and should not be construed as limiting the intent and scope of the invention.

1. A composite bolster scaffold comprising:
a tissue scaffold; and
an anchor bolster securely coupled to the tissue scaffold, wherein the anchor bolster comprises a first surface, a second surface, and a first thickness between the first surface and the second surface.

2. The bolster scaffold of claim 1, wherein the tissue scaffold is at least one of: an acellular dermal matrix, an acellular adipose matrix, or an acellular muscle matrix.

3. The bolster scaffold of claim 1, wherein the tissue scaffold is a formed three-dimensional tissue scaffold.

4. The bolster scaffold of claim 1, wherein the anchor bolster comprises a non-biologic biocompatible material.

5. The bolster scaffold of claim 1, wherein the anchor bolster comprises at least one anchor fixation point.

6. The bolster scaffold of claim 5, wherein the anchor fixation point is an opening extending through the first surface and the second surface.

7. The bolster scaffold of claim 5, wherein the anchor fixation point comprises a second thickness less than the first thickness.

8. The bolster scaffold of claim 5, wherein the anchor fixation point comprises a rim having a third thickness greater than the first thickness.

9. The bolster scaffold of claim 1, wherein the anchor bolster is shaped as a tab.

10. The bolster scaffold of claim 1, wherein the anchor bolster is shaped as a ribbon, wherein the ribbon follows a contour of a perimeter of the tissue scaffold.

11. A method of using a composite bolster-scaffold comprising:
selecting a tissue scaffold securely coupled to an anchor bolster;
positioning the tissue scaffold proximate to an anatomic structure;
attaching at least one surgical anchor to the anchor bolster; and
securing the anchor to the anatomic structure.

12. The method of claim 11, wherein the tissue scaffold is at least one of: an adipose tissue scaffold, a dermal tissue scaffold, or a muscle tissue scaffold.

13. The method of claim 11, wherein the tissue scaffold is a three-dimensional formed acellular tissue matrix scaffold.

14. The method of claim 11, wherein the anchor bolster comprises a non-biologic biocompatible material.

15. The method of claim 11, wherein the anchor bolster comprises at least one anchor fixation point and wherein the at least one surgical anchor is attached to an anchor fixation point of the at least one anchor fixation points.

16. The method of claim 15, wherein the anchor fixation point is an opening extending through a first surface and a second surface of the anchor bolster.

17. The method of claim 15, wherein the anchor fixation point comprises a second thickness less than a first thickness of the anchor bolster.

18. The method of claim 15, wherein the anchor bolster comprises a rim having a third thickness greater than a first thickness between a first surface and a second surface of the anchor bolster.

19. The method of claim 11, wherein the anchor bolster is shaped as a tab.

20. The method of claim 11, wherein the anchor bolster is shaped as a ribbon, wherein the ribbon follows a contour of a perimeter of the tissue scaffold.
